(19) United States
(12) Patent Application Publication
(10) Pub. No.: US 2017/0028183 A1
(43) Pub. Date: Feb. 2, 2017

(54) INGESTIBLE CAPSULE

(71) Applicant: Mayo Foundation for Medical Education and Research, Rochester, MN (US)

(72) Inventors: Heidi Nelson, Rochester, MN (US); Nicholas Chia, Rochester, MN (US)

(73) Assignee: Mayo Foundation for Medical Education and Research, Rochester, MN (US)

(21) Appl. No.: 15/302,373

(22) PCT Filed: Apr. 8, 2015
(86) PCT No.: PCT/US15/24938

§ 371 Date: Oct. 6, 2016

Related U.S. Application Data
(60) Provisional application No. 61/976,626, filed on Apr. 8, 2014.

(51) Int. Cl. A61M 31/00; A61K 9/48; A61K 9/00; A61K 35/741

(52) U.S. Cl. CPC A61M 31/002; A61K 35/741; A61K 9/4808; A61K 9/0053; A61M 2210/1053; A61M 2210/106; A61M 2210/1064; A61K 2035/11

(57) ABSTRACT

This document provides ingestible capsule devices and methods for using the ingestible capsule devices to treat a patient. For example, this document provides ingestible capsule devices that can deliver probiotics with high viability to a focal gastrointestinal target site using a time-based delivery system.

### CROSS-REFERENCE TO RELATED APPLICATIONS

This application claims the benefit of U.S. Provisional Application Ser. No. 61/976,626, filed Apr. 8, 2014. The disclosure of that prior application is considered part of (and is incorporated by reference in) the disclosure of this application.

### BACKGROUND

1. Technical Field

This document relates to ingestible capsule devices and methods for using the ingestible capsule devices to treat a patient. For example, this document relates to ingestible capsule devices that can deliver probiotics with high viability to a focal gastrointestinal target site using a time-based delivery system.

2. Background Information

Probiotics are food supplements that contain live microorganisms which when administered in adequate amounts confer a health benefit on the recipient. In addition, the health benefits of diverse commensal microbial populations are becoming increasingly recognized. Probiotics are also gathering interest due to a greater awareness of the limitations and complications of antibiotic usage, such as drug resistance and superinfections like Clostridium difficile. For example, clinical trials have demonstrated that vaginal probiotics can reduce the rate of urinary tract infections. Further, orally administered probiotics can potentially: 1) prevent Clostridium difficile-associated diarrhea when administered with antibiotics, 2) eradicate Helicobacter pylori together with antibiotics, 3) prevent and reduce the duration of diarrhea in children with gastroenteritis, and 4) restore normal vaginal microbial populations.

Despite many trial successes and the general popularity of probiotics, the widespread adoption of probiotics into medical practice has met with variable success in clinical trials and limited enthusiasm by providers. One of the greatest impediments to the advancement of probiotics has been the lack of more precise estimates of how probiotics get to and impact target microbial communities within the patient. To be effective, a probiotic must reach the right target (e.g., stomach, small intestine, colon) at the right time. For example, the delivery timing includes the intestinal transit times that can be effected by various patient conditions from normal, to the slow transit resulting from constipation, or the fast transit resulting from diarrhea. In addition, a further challenge to the delivery of probiotics to a focal gastrointestinal (GI) target site is the need for maintaining probiotic viability through the harsh intestinal environment.

### SUMMARY

This document provides ingestible capsule devices and methods for using the ingestible capsule devices to treat a patient. For example, this document provides ingestible capsule devices that can deliver probiotics with high viability to a focal gastrointestinal target site using a time-based delivery system. The devices provided herein are inert to bacteria and can carry a large payload of therapeutic material, such as a payload of probiotics, for example. In some embodiments, the capsule devices provided herein have multiple chambers that can be individually timed to deliver multiple boluses according to calculated or measured estimates of target specific transit times.

In general, one aspect of this document features a method for treating a human patient. The method comprises administering a therapeutic substance comprising a probiotic to the patient such that the patient ingests a capsule containing the therapeutic substance, and such that the therapeutic substance is released from the capsule in the GI tract of the patient after a pre-selected time has elapsed.

Such a method for treating a human patient may optionally include one or more of the following features. The capsule may substantially prevents any release of the therapeutic substance prior to after the pre-selected time has elapsed. The therapeutic substance may be released from the capsule in the patient's stomach. The therapeutic substance may be released from the capsule in the patient's small intestine. The therapeutic substance may be released from the capsule in the patient's colon. The pre-selected time may be between about 2 hours to about 4 hours after the patient ingests the capsule. The pre-selected time may be between about 4 hours to about 6 hours after the patient ingests the capsule. The pre-selected time may be between about 6 hours to about 8 hours after the patient ingests the capsule. The pre-selected time may be between about 8 hours to about 10 hours after the patient ingests the capsule. The pre-selected time may be greater than about 10 hours after the patient ingests the capsule. The therapeutic substance may be a first therapeutic substance, the probiotic may be a first probiotic, and the pre-selected time may be a first pre-selected time; and the method may further comprise administering a second therapeutic substance comprising a second probiotic to the patient such that the second therapeutic substance is released from the capsule in the GI tract of the patient after a second pre-selected time has elapsed. In some embodiments, the first therapeutic substance and the second therapeutic substance may be different substances. The first pre-selected time may be different from the second pre-selected time. The first probiotic may be different from the second probiotic. The method may further comprise administering a third therapeutic substance comprising a third probiotic to the patient such that the third therapeutic substance is released from the capsule in the GI tract of the patient after a third pre-selected time has elapsed. The first, second, and third pre-selected times may each be distinctly different times.

Particular embodiments of the subject matter described in this document can be implemented to realize one or more of the following advantages. In some embodiments, the capsule devices provided herein can facilitate a method for effective dispensation of therapeutic materials from an ingested capsule to a patient. For example, in some embodiments the capsule devices provided herein can be used to deliver probiotics to treat various patient conditions as well as complications pertaining to antibiotic usage such as drug resistance and superinfections like Clostridium difficile. The capsule devices provided herein can protect the contents of the capsule by standing up to the harsh environment of the GI tract as the capsule travels toward the target release site. As a result, the contents of the capsule are not released or diluted prior to a pre-designated release time at a target site in the GI tract of the patient. Accordingly, the therapeutic contents of the capsule can be released at the target site at a desired potency for enhanced efficacy. Further, a time-based release approach is used such that the therapeutic contents of the capsule can be released at a pre-selected target site within the GI tract of the patient for enhanced efficacy.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1A is an exploded perspective view of an ingestible capsule device in accordance with some embodiments provided herein.

FIG. 1B is a transparent perspective view showing the ingestible capsule device of FIG. 1A in an assembled configuration.

FIG. 2A is a side view of another ingestible capsule device in accordance with some embodiments provided herein.

FIG. 2B is a top view of the ingestible capsule device of FIG. 2A.

FIG. 2C is a top view of the ingestible capsule device of FIG. 2A shown partially open such that the contents of the capsule device are released.

FIG. 3 is a flowchart of a method for treating a patient using an ingestible capsule device in accordance with some embodiments provided herein.

Like reference numbers represent corresponding parts throughout.

### DETAILED DESCRIPTION

This document provides ingestible capsule devices and methods for using the ingestible capsule devices to treat a patient. For example, this document provides ingestible capsule devices that can deliver probiotics with high viability to a focal gastrointestinal target site using a time-based delivery system.

The devices and methods provided herein can also be used to delivery other therapeutic materials in addition to probiotics. In one such non-limiting example, a dye to be used as part of a study or imaging analysis can be delivered using the devices and methods provided herein. The dye could be used, for example, to examine blood flow to the liver in order to study vessel structure in liver failure. Other uses for the devices and methods provided herein are also envisioned.

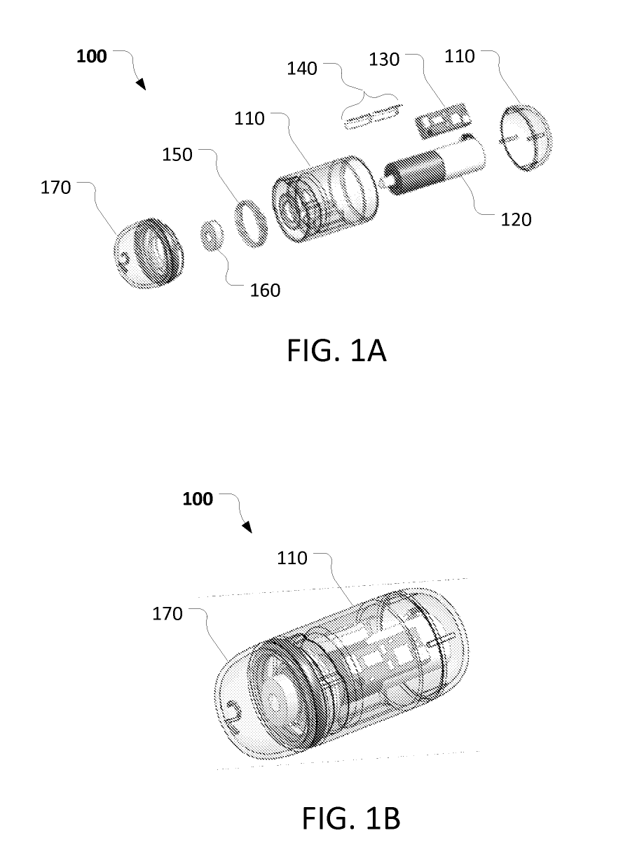

With reference to FIGS. 1A and 1B, an example ingestible capsule device 100 includes a housing 110 that is releasably coupleable with a delivery capsule 170. Between housing 110 and delivery capsule 170 is an open interior space configured for containment of a therapeutic material that can be released to a subject (e.g., released in a GI tract of a human patient) upon separation of delivery capsule 170 from housing 110. In some embodiments, an injection port may be included on delivery capsule 170 or housing 110 through which therapeutic material can be loaded into delivery capsule 170 while delivery capsule is coupled to housing 110.

Housing 110 and delivery capsule 170 combine to define the outer shape and size of ingestible capsule device 100. In some embodiments, the outer dimensions of ingestible capsule device 100 do not exceed 26 mm by 11 mm. In some embodiments, the weight of ingestible capsule device 100 is 3.7 grams or less. In some embodiments, a gasket is included between housing 110 and delivery capsule 170 to protect the therapeutic material contents of the interior space of delivery capsule 170.

Housing 110 and delivery capsule 170 are comprised of materials that are inert to therapeutic materials including, but not limited to, probiotics. Further, housing 110 and delivery capsule 170 are comprised of materials that are inert to humans, including the GI tract of humans. Accordingly, housing 110 and delivery capsule 170 can be ingested by a human patient without substantially deteriorating while in the patient's GI tract.

In general, ingestible capsule device 100 can be programmed to open after a pre-determined period of time. That is, ingestible capsule device 100 includes a timer and when the timer determines that a pre-determined period of time has elapsed, housing 110 and delivery capsule 170 will separate from each other to expose or release the contents of delivery capsule 170. In one example implementation, the pre-determined time period can be about 8 hours. For example, the timer can be started and then the patient can ingest the capsule device 100 soon thereafter. After about 8 hours capsule device 100 will likely be positioned in the patient's small intestine, for example. After expiry of the 8 hours, housing 110 and delivery capsule 170 will therefore separate from each other to release the contents of delivery capsule 170 within the patient's small intestine. It should be understood that the pre-determined time period after which the capsule device 100 will open can be longer or shorter than 8 hours. For example, in some embodiments the pre-determined time period may be about 1 hour, about 2 hours, about 3 hours, about 4 hours, about 5 hours, about 6 hours, about 7 hours, about 9 hours, about 10 hours, about 11 hours, about 12 hours, or greater than 12 hours. The pre-determined time period can also include fractions of hours (e.g., 4 hours and 30 minutes) and even seconds.

Still referring to FIGS. 1A and 1B, in some embodiments housing 110 contains multiple components that define and/or control the releasably coupleable relationship between delivery capsule 170 and housing 110. For example, in some embodiments such components can include a motor 120, a control system 130, one or more batteries 140, a spring 150, and a latch 160.

In some embodiments, latch 160 is configured to releasably couple with delivery capsule 170. For example, in the depicted embodiment latch 160 is elliptical and delivery capsule 170 includes a complementary shaped elliptical aperture that is slightly larger than the elliptical latch 160. When delivery capsule 170 is fully seated on housing 110, latch 160 projects beyond the complementary elliptically shaped aperture and into the interior space of delivery capsule 170. When the elliptical latch 160 is aligned with the complementary elliptically shaped aperture of delivery capsule 170, delivery capsule 170 can separate from being coupled with latch 160. Spring 150 can then urge delivery capsule 170 to linearly separate from housing 110. However, when the elliptical latch 160 is rotated out of alignment with the complementary elliptically shaped aperture of delivery capsule 170, delivery capsule 170 is latched to housing 110. While in the depicted embodiment latch 160 and the corresponding aperture in delivery capsule 170 are elliptical, it should be understood that other shapes, profiles, features, and mechanisms can be used to interlock latch 160 with delivery capsule 170.

In some embodiments, a driveshaft of motor 120 is coupled (e.g., keyed, adhered, etc.) to latch 160 so that motor 120 can rotate latch 160 in the manner aforementioned. That is, upon expiry of the pre-determined time period, motor 120 can automatically actuate such that motor 120 rotates the elliptical latch 160 into alignment with the complementary elliptically shaped aperture of delivery capsule 170. Then spring 150 can force delivery capsule 170 to become separated from housing 110 such that the contents of the interior space of delivery capsule 170 are exposed or released therefrom.

In some embodiments, control system 130 can perform the timing function and the actuation of motor 120. For example, control system 130 can be a microcontroller (including memory for storing executable instructions) having functionalities such as, but not limited to, a programmable timer and motor driver circuitry for driving motor 120. In some embodiments, one or more sensors (e.g., Hall Effect sensors) are included in control system 130. Such sensors can be used to for various purposes including, but not limited to, programming the pre-determined period of time and starting the timer prior to ingestion. In some embodiments, the pre-determined period of time is preprogrammed into control system 130 and is not readily alterable. In some such embodiments, ingestible capsule devices 100 having differing pre-established pre-determined time periods are obtainable from an inventory of devices 100. In some embodiments, control system 130 also includes a user interface. In some embodiments, the user interface includes devices such as, but not limited to, one or more LED indicator lights, an LCD display, input buttons, and the like.

One or more batteries 140 can provide the energy to operate control system 130 and to actuate motor 120. In some embodiments, two batteries 140 are included. In some such embodiments, one of the two batteries 140 is a low-drain type for operating the control system 130 prior to actuation of motor 120, and the second of the two batteries 140 is a high-drain type for actuating motor 120. However, in some embodiments a single battery 140 or more than two batteries 140 can be used.

In some embodiments, a fuse-like filament can be used to hold ingestible capsule device 100 closed. When the time has expired such that the contents of ingestible capsule device 100 should be released, heat generated by energy from batteries 140 can melt the filament and ingestible capsule device 100 can spring open.

In some embodiments, a watch-like timer mechanism can be used to hold ingestible capsule device 100 closed. When the time has expired such that the contents of ingestible capsule device 100 should be released, the timer can allow ingestible capsule device 100 to spring open.

In some embodiments, a friction clamp can be used to hold ingestible capsule device 100 closed. Using a piezo actuator or nitinol wire, the system clamps onto the capsule. When release is desired, the actuator is activated, and un-clamps the capsule, allowing it to fall away.

In some embodiments, ingestible capsule device 100 can include one or more pressurized reservoirs. Using a miniature valve, the system would be pre-pressurized, and when the desired release time occurs, the valve is opened and the pressure ejects the capsule.

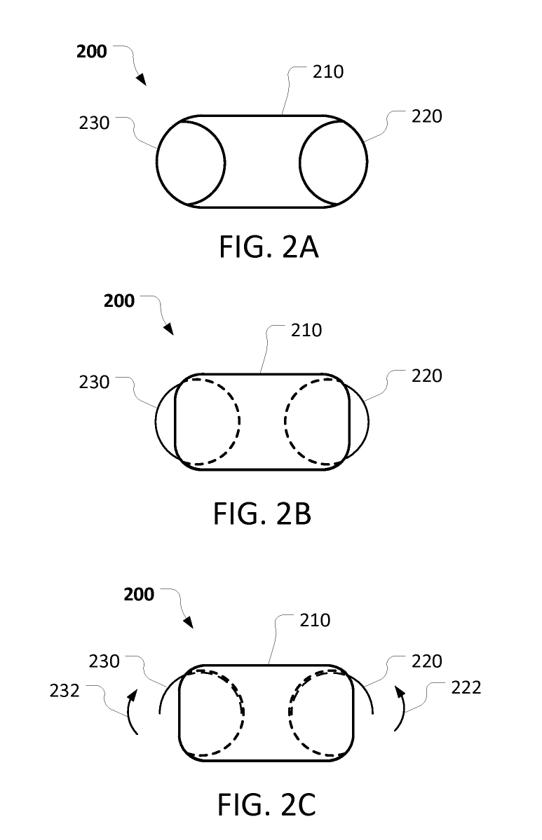

With reference to FIGS. 2A-2C, another example embodiment of an ingestible capsule device 200 can include multiple chambers for containing and releasing one or more types of therapeutic substances. Ingestible capsule device 200 includes a housing 210, a first delivery capsule 220, and a second delivery capsule 230. In some implementations, first delivery capsule 220 and second delivery capsule 230 are each loaded with the same therapeutic substance. Alternatively, in some implementations first delivery capsule 220 and second delivery capsule 230 are loaded with different therapeutic substances.

In the depicted embodiment, first delivery capsule 220 and second delivery capsule 230 are pivotable in relation to housing 210, so as to release the interior contents of first delivery capsule 220 and second delivery capsule 230. That is, first delivery capsule 220 can pivot as indicated by arrow 222, and second delivery capsule 230 can pivot as indicated by arrow 232. First delivery capsule 220 and second delivery capsule 230 can pivot fully open to be entirely adjacent to housing 210. In alternative embodiments, other types of mechanisms and movements can be used to open the interior spaces of first delivery capsule 220 and second delivery capsule 230. For example, first delivery capsule 220 and second delivery capsule 230 can be separated from housing 210 using a mechanism that is analogous to the mechanism described above in reference to ingestible capsule device 100.

In some embodiments, first delivery capsule 220 and second delivery capsule 230 simultaneously open to release materials contained therein. However, in some embodiments first delivery capsule 220 and second delivery capsule 230 are separately operable, and can be pivotably opened independently of each other and at different times if programmed to do so.

While ingestible capsule device 200 includes two delivery capsules, in some embodiments three or more delivery capsules can be included. In some such embodiments, the three or more delivery capsules can be opened simultaneously or at different time intervals as desired.

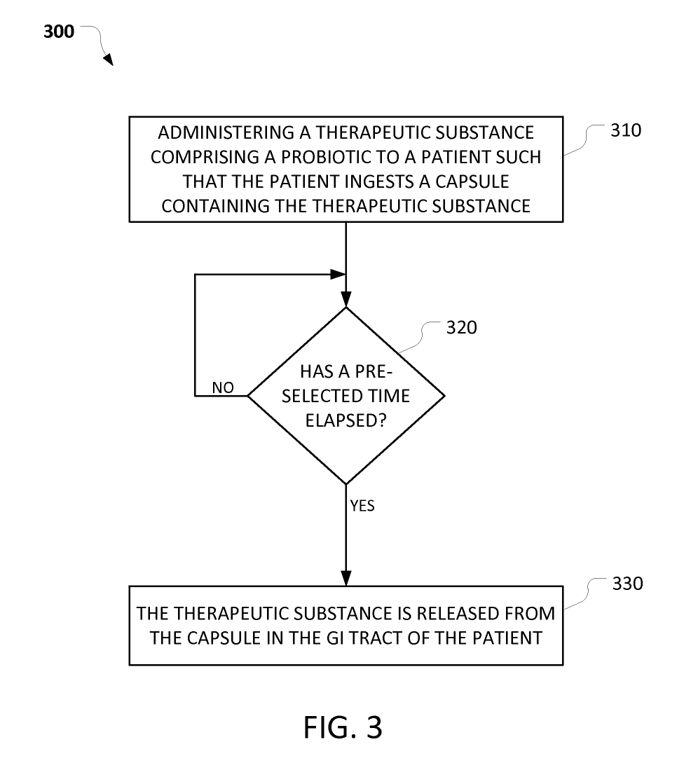

With reference to FIG. 3, a flow chart is provided of an example method for treating a patient using an ingestible capsule device in accordance with some embodiments provided herein. Step 310 comprises administering a therapeutic substance (a therapeutic substance comprising a probiotic, for example) to the patient such that the patient ingests a capsule containing the therapeutic substance. The capsule can be configured, for example, like the ingestible capsule devices 100 or 200 described above. After ingestion of the capsule at step 310, the ingestible capsule device remains closed for a predetermined time period while traveling within the GI tract of the patient.

At step 320, the controller of the capsule determines whether the pre-determined time period has elapsed. If the pre-determined time period has not elapsed, the capsule remains closed. When the pre-determined time period has elapsed, the method proceeds to step 330.

At step 330, the therapeutic substance is released from the capsule. The therapeutic substance is thereby released at a desired location within the GI tract of the patient after the pre-selected time has elapsed.

The devices and methods provided herein are well-suited to administer many different types of substances to the GI tract. Non-limiting examples of such substances may include: Hydrocortisone, Budenoside, Prednisolone, Sulfaselazine, Olsalazine, Mesalazine, Belsalazide, digestive enzyme supplements, 5-Flourouracil, NSAIDS, steroids, insulin, anti-inflammatory drugs, antihypertensive and antianginal drugs, antineoplastic drugs, peptides and proteins, nitroglycerin and corticosteroids, antiarthritic and antiasthamatic drugs, 5-ASA, salicylic acid, Dexamethasone/Prednisolone, Vasopressin insulin, 5-(6) carboxy fluorescein (CF), sodium diclofenace, Indomethacin, Paracetamol, Ropivacaine, Paracetamol, and Ropivacaine.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A method of treating a human patient, the method comprising:
administering a therapeutic substance comprising a probiotic to the patient such that the patient ingests a capsule containing the therapeutic substance and such that the therapeutic substance is released from the capsule in the GI tract of the patient after a pre-selected time has elapsed.

2. The method of claim 1, wherein the capsule substantially prevents any release of the therapeutic substance prior to after the pre-selected time has elapsed.

3. The method of claim 1, wherein the therapeutic substance is released from the capsule in the patient's stomach.

4. The method of claim 1, wherein the therapeutic substance is released from the capsule in the patient's small intestine.

5. The method of claim 1, wherein the therapeutic substance is released from the capsule in the patient's colon.

6. The method of claim 1, wherein the pre-selected time is between about 2 hours to about 4 hours after the patient ingests the capsule.

7. The method of claim 1, wherein the pre-selected time is between about 4 hours to about 6 hours after the patient ingests the capsule.

8. The method of claim 1, wherein the pre-selected time is between about 6 hours to about 8 hours after the patient ingests the capsule.

9. The method of claim 1, wherein the pre-selected time is between about 8 hours to about 10 hours after the patient ingests the capsule.

10. The method of claim 1, wherein the pre-selected time is greater than about 10 hours after the patient ingests the capsule.

11. The method of claim 1, wherein the therapeutic substance is a first therapeutic substance, wherein the probiotic is a first probiotic, and wherein the pre-selected time is a first pre-selected time, and
further comprising administering a second therapeutic substance comprising a second probiotic to the patient such that the second therapeutic substance is released from the capsule in the GI tract of the patient after a second pre-selected time has elapsed.

12. The method of claim 11, wherein the first therapeutic substance and the second therapeutic substance are different substances.

13. The method of claim 11, wherein the first pre-selected time is different from the second pre-selected time.

14. The method of claim 11, wherein the first probiotic is different from the second probiotic.

15. The method of claim 11, further comprising administering a third therapeutic substance comprising a third probiotic to the patient such that the third therapeutic substance is released from the capsule in the GI tract of the patient after a third pre-selected time has elapsed.

16. The method of claim 15, wherein the first, second, and third pre-selected times are each distinctly different times.
